(19) United States
(12) Patent Application Publication
(10) Pub. No.: US 2011/0065632 A1
(43) Pub. Date: Mar. 17, 2011

(54) PHARMACEUTICAL COMPOSITIONS OF SOMATOSTATIN-DOPAMINE CONJUGATES

(76) Inventors: Zheng Xin Dong, Holliston, MA (US); Jundong Zhang, Newton, MA (US)

(21) Appl. No.: 12/991,348

(22) PCT Filed: May 12, 2009
(86) PCT No.: PCT/US09/02943

§ 371 Date: Nov. 5, 2010

Related U.S. Application Data
(60) Provisional application No. 61/127,584, filed on May 14, 2008.
(60) Provisional application No. 61/200,058, filed on Nov. 24, 2008.

(51) Int. Cl. A61K 38/31; C07K 14/655; A61P 3/04; A61P 1/00; A61P 3/10; A61P 35/00; A61P 1/12; A61P 3/06

(52) U.S. Cl. 514/4.8; 530/311; 514/11.1; 514/7.1

(57) ABSTRACT

The present invention is directed to improvements in compositions containing a somatostatin-dopamine conjugate which retains both somatostatin and dopamine activity in vivo, methods for preparing such compositions, and method of using such compositions to treat mammals. In particular, the present invention relates to a pharmaceutical composition comprising Dop2-DLys(Dop2)-cyclo[Cys-Tyr-DTrp-Lys-Abu-Cys]-Thr-NH₂ (SEQ ID NO: 1), in which the somatostatin-dopamine conjugate precipitates in vivo at physiological pH to form an in situ deposit that is slowly dissolved and released into the body fluid and bloodstream. The present invention may further comprise an organic component such as dimethylacetamide (DMA) or polyethylene glycol with an average molecular weight of 400 (PEG400).

### BACKGROUND OF THE INVENTION

The present invention relates to improvements in compositions containing a somatostatin-dopamine conjugate which retains both somatostatin and dopamine activity in vivo, methods for preparing such compositions, and method of using such compositions to treat mammals. In particular, the present invention relates to a pharmaceutical composition comprising Dop2-DLys(Dop2)-cyclo[Cys-Tyr-DTrp-Lys-Abu-Cys]-Thr-NH₂ (SEQ ID NO:1), in which the somatostatin-dopamine conjugate precipitates in vivo at physiological pH to form an in situ deposit that is slowly dissolved and released into the body fluid and bloodstream. The present invention may further comprise an organic component such as dimethylacetamide (DMA) or polyethylene glycol with an average molecular weight of 400 (PEG400).

Dopamine is a catecholamine neurotransmitter that has been implicated in the pathogenesis of both Parkinson's disease and schizophrenia. Dopamine and related molecules have been shown to inhibit the growth of several types of malignant tumors in mice, and this activity has been variously attributed to inhibition of tumor-cell proliferation, stimulation of tumor immunity as well as effects on melanin metabolism in malignant melanomas. Recent studies demonstrated the presence of D2 dopamine receptors on endothelial cells. Dopamine has recently been reported to strongly and selectively inhibit at non-toxic levels the vascular permeabilizing and angiogenic activities of VPF/VEGF.

Somatostatin (SS), a tetradecapeptide has been shown to have potent inhibitory effects on various secretory processes in tissues such as pituitary, pancreas and gastrointestinal tract. SS also acts as a neuromodulator in the central nervous system. These biological effects of SS, all inhibitory in nature, are elicited through a series of G protein coupled receptors, of which five different subtypes have been characterized (SSTR-1-SSTR-5). These five subtypes have similar affinities for endogenous SS ligands, but have differing distributions in various tissues. Somatostatin binds to the five distinct receptor (SSTR) subtypes with relatively high and equal affinity for each subtype.

There is evidence that SS regulates cell proliferation by arresting cell growth via SSTR-1, -2, -3, -4, and -5 subtypes, and/or by inducing apoptosis via SSTR-3 subtype. SS and various analogues have been shown to inhibit normal and neoplastic cell proliferation in vitro and in vivo via specific SS receptors (SSTR's) and possibly different postreceptor actions. In addition, there is evidence that distinct SSTR subtypes are expressed in normal and neoplastic human tissues, conferring different tissue affinities for various SS analogues and variable clinical response to their therapeutic effects.

Binding to different types of somatostatin receptor subtypes is associated with the treatment of various conditions and/or diseases. For example, the inhibition of growth hormone has been attributed to the somatostatin type-2 receptor (“SSTR-2”), while the inhibition of insulin has been attributed to the somatostatin type-5 receptor (“SSTR-5”). Activation of types 2 and 5 have been associated with growth hormone suppression and more particularly growth hormone secreting adenomas (acromegaly) and thyroid stimulating hormone (TSH) secreting adenomas. Activation of type 5 but not type 2 receptor has been associated with treating prolactin secreting adenomas. Other indications associated with activation of the somatostatin receptor subtypes include inhibition of insulin and/or glucagon for treating diabetes mellitus, angiopathy, proliferative retinopathy, dawn phenomenon, and nephropathy; inhibition of gastric acid secretion for treating peptic ulcers, enterocutaneous and pancreaticocutaneous fistula, irritable bowel syndrome, Dumping syndrome, watery diarrhea syndrome, AIDS related diarrhea, chemotherapy-induced diarrhea, acute or chronic pancreatitis and gastrointestinal hormone secreting tumors; treatment of cancer such as hepatoma; inhibition of angiogenesis; treatment of inflammatory disorders such as arthritis; retinopathy; chronic allograft rejection; angioplasty; preventing graft vessel and gastrointestinal bleeding. Preferably, a somatostatin analog is selective for the specific somatostatin receptor subtype or subtypes responsible for the desired biological response to reducing interaction with other receptor subtypes which could lead to undesirable side effects or loss of efficacy.

Somatostatin and its receptors (SSTR-1 to SSTR-5) are expressed in normal human parafollicular C cells and medullary thyroid carcinoma (MTC). MTC is a tumor originating from thyroid parafollicular C cells that produce calcitonin (CT), somatostatin, and several other peptides. It was recently demonstrated that SS and SSTR's are expressed in human MTC, and SS and SS analogues were shown to induce a decrease in plasma CT levels and provide symptomatic improvement in MTC patients. Another recent study has shown that SS and SS analogues, in particular, SSTR-1 and SSTR-2, can inhibit the proliferation of tumor cells, suggesting that specific SSTR subtypes can function in MTC cell growth regulation. The development and characterization of SSTR subtype analogues that selectively effect MTC cell growth is useful for clinical and therapeutic applications.

### SUMMARY OF THE INVENTION

The present invention provides a pharmaceutical composition comprising a dopamine-somatostatin conjugate. Particularly preferred is the following dopamine-somatostatin conjugate, which is referred to hereinafter as “Example 1”: Dop2-DLys(Dop2)-cyclo[Cys-Tyr-DTrp-Lys-Abu-Cys]-Thr-NH₂ (SEQ ID NO:1), or a pharmaceutically acceptable salt thereof, wherein the formulation of said composition provides for superior manufacturing, administration, pharmacokinetic and pharmacodynamic properties, as well as attenuated negative side effects. Example 1's molecular structure is:

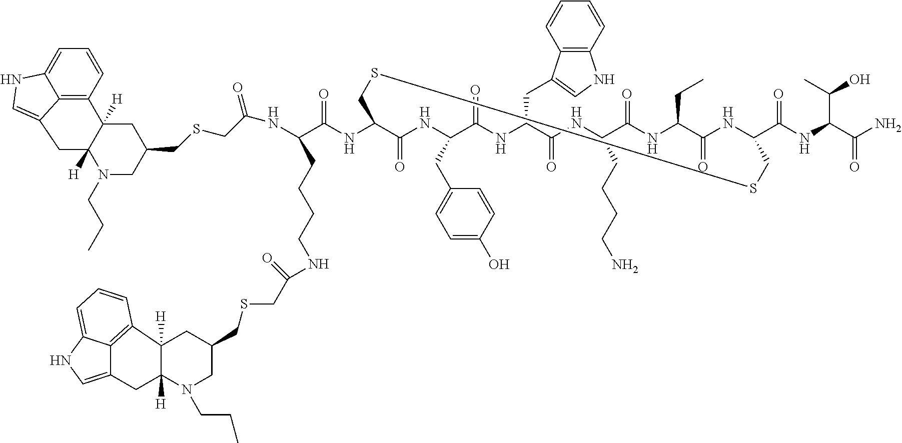

In preferred features, the invention provides for a pharmaceutical composition in which the dopamine-somatostatin conjugate precipitates in vivo at physiological pH to form an in situ deposit that is slowly dissolved and released into the body fluid and bloodstream. The invention may be summarized in the following paragraphs (1) through (38), below, as well as the claims. Accordingly:
(1) In one aspect, the present invention is directed to a pharmaceutical composition of a clear aqueous solution, or a gel or a semi-solid, comprising a somatostatin-dopamine conjugate, or a pharmaceutically acceptable salt thereof, in which the somatostatin-dopamine conjugate forms a precipitate after subcutaneous or intramuscular administration to a subject.
(2) The pharmaceutical composition according to paragraph (1), wherein said somatostatin-dopamine conjugate is Example 1, i.e., Dop2-DLys(Dop2)-cyclo[Cys-Tyr-DTrp-Lys-Abu-Cys]-Thr-NH₂ (SEQ ID NO:1).
(3) The pharmaceutical composition according to paragraph (2), further comprising an organic component.
(4) The pharmaceutical composition
(5) The pharmaceutical composition according to paragraph (3), wherein said organic component increases solubility of the somatostatin-dopamine conjugate in an aqueous solution or decreases viscosity of a gel or a semi-solid.
(6) The pharmaceutical composition according to paragraph (4), wherein said organic component is an organic polymer.
(7) The pharmaceutical composition according to paragraph (5), wherein said organic polymer is polyethylene glycol (PEG).
(8) The pharmaceutical composition according to paragraph (6), wherein said PEG is selected from the group consisting of PEG300, PEG400 and PEG1750.
(9) The pharmaceutical composition according to paragraph (8), wherein said somatostatin-dopamine conjugate is dissolved in 20% PEG400 water solution at the concentration of about 30% (w/v).
(10) The pharmaceutical composition according to paragraph (8), wherein said somatostatin-dopamine conjugate is dissolved in 5% DMA water solution at the concentration of about 200 mg/mL.
(11) The pharmaceutical composition according to paragraph (8), wherein said somatostatin-dopamine conjugate is dissolved in 5% PEG400 water solution at the concentration of about 200 mg/mL.
(12) The pharmaceutical composition according to any one of paragraphs (1)-(3), wherein said somatostatin-dopamine conjugate is dissolved in water at the concentration range of about 15-30% (w/v).
(13) The pharmaceutical composition according to paragraph (12), wherein said somatostatin-dopamine conjugate is dissolved in water at the concentration of about 15% (w/v).
(14) The pharmaceutical composition according to paragraph (12), wherein said somatostatin-dopamine conjugate is dissolved in water at the concentration of about 30% (w/v).
(15) The pharmaceutical composition according to paragraph (4), wherein said organic component is an organic solvent.
(16) The pharmaceutical composition according to paragraph (15), wherein said organic solvent is an amide
(17) The pharmaceutical composition according to paragraph (16), wherein said amide is dimethylacetamide (DMA).
(18) The pharmaceutical composition according to paragraph (4), wherein said organic component is an alcohol.
(19) The pharmaceutical composition according to paragraph (18), wherein said alcohol is selected from the group consisting of ethanol, propanol and propylene glycol.
(20) The pharmaceutical composition according to paragraph (4), wherein said organic component is a sugar.
(21) The pharmaceutical composition according to paragraph (4), wherein said organic component is a cyclodextrin.
(22) The pharmaceutical composition according to paragraph (21), wherein said cyclodextrin is selected from the group consisting of hydroxypropyl-cyclodextrin and sulfobutylether-cyclodextrin.
(23) The pharmaceutical composition according to paragraph (4), wherein said organic component is a phospholipid.
(24) The pharmaceutical composition according to paragraph (23), wherein said phospholipid is selected from the group consisting of hydrogenated soy phosphatidylcholine, distearoylphosphatidylglycerol, 1-dimyristoylphosphatidylcholine, and 1-dimyristoylphosphatidylglycerol.
(25) The pharmaceutical composition according to paragraph (4), wherein said organic component is a water-soluble organic solvent.
(26) The pharmaceutical composition according to paragraph (25), wherein said water-soluble organic solvent is selected from the group consisting of PEG300, ethanol, propylene glycol, glycerin, N-methyl-2-pyrrolidone, dimethylacetamide, and dimethylsulfoxide.
(27) The pharmaceutical composition according to paragraph (4), wherein said organic component is a non-ionic surfactant.
(28) The pharmaceutical composition according to paragraph (27), wherein said non-ionic surfactant is selected from the group consisting of Cremophor EL, Cremophor RH 40, Cremophor RH 60, d-tocopherol polyethylene glycol 1000 succinate, polysorbate 20, polysorbate 80, sorbitan monooleate, poloxamer 407, Labrafil M-1944CS, Labrafil M-2125CS, Labrasol, Gellucire 44/14, Softigen 767, and mono- and di-fatty esters of PEG300, PEG400 or PEG1750.
(29) The pharmaceutical composition according to paragraph (4), wherein said organic component is an ester.
(30) The pharmaceutical composition according to paragraph (29), wherein said ester is polyglycol ester.
(31) The pharmaceutical composition according to any one of paragraphs (1) to (30), wherein the somatostatin-dopamine conjugate is present in an aqueous solution with pH between 1.0 and 10.5, preferably between 3 and 8, and more preferably between 5 and 6.
(32) The pharmaceutical composition according to any one of paragraphs (1) to (31), wherein the somatostatin-dopamine conjugate is present in a concentration of about from 0.0001 to 500 mg/mL, preferable about from 0.1 to 300 mg/mL.
(33) The pharmaceutical composition according to any one of paragraphs (1) to (32), further comprising a preservative.
(34) The pharmaceutical composition according to paragraph (33), wherein said preservative is selected from the group consisting of m-cresol, phenol, benzyl alcohol, and methyl paraben.
(35) The pharmaceutical composition according to paragraph (34), wherein said preservative is present in a concentration from 0.01 mg/mL to 100 mg/mL.
(36) The pharmaceutical composition according to any one of paragraphs (1) to (35), further comprising an isotonic agent.
(37) The pharmaceutical composition according to paragraph (36), wherein said isotonic agent is present in a concentration from 0.01 mg/mL to 100 mg/mL.
(38) The pharmaceutical composition according to any one of paragraphs (1) to (37), further comprising a stabilizer.
(39) The pharmaceutical composition according to paragraph (38), wherein said stabilizer is selected from the group consisting of imidazole, arginine and histidine.
(40) The pharmaceutical composition according to any one of paragraphs (1) to (39), further comprising a surfactant.
(41) The pharmaceutical composition according to any one of paragraphs (1) to (40), further comprising a chelating agent.
(42) The pharmaceutical composition according to any one of paragraphs (1) to (41), further comprising a buffer.
(43) The pharmaceutical composition according to paragraph (42), wherein said buffer is selected from the group consisting of Tris, ammonium acetate, sodium acetate, glycine, aspartic acid, and Bis-Tris.
(44) The pharmaceutical composition according to any one of paragraphs (1) to (43), further comprising a divalent metal.
(45) The pharmaceutical composition according to paragraph (44), wherein said divalent metal is zinc.

Although the preferred embodiment of the present invention is directed to Example 1 as the somatostatin-dopamine conjugate which retains both somatostatin and dopamine activity in vivo, the present invention is in no way limited to Example 1. The somatostatin-dopamine conjugates of the present invention includes, for example, all those somatostatin-dopamine conjugates which retain both somatostatin and dopamine activity in vivo, as disclosed in the Applicant's prior international publication numbers published as WO 2004/091490 and WO 02/100888. These publications are herein incorporated by reference to the same extent as if the disclosure of each independent publication was explicitly provided herein.

The following somatostatin-dopamine conjugates from these publications may also be advantageously employed to constitute the pharmaceutical compositions of the present invention:

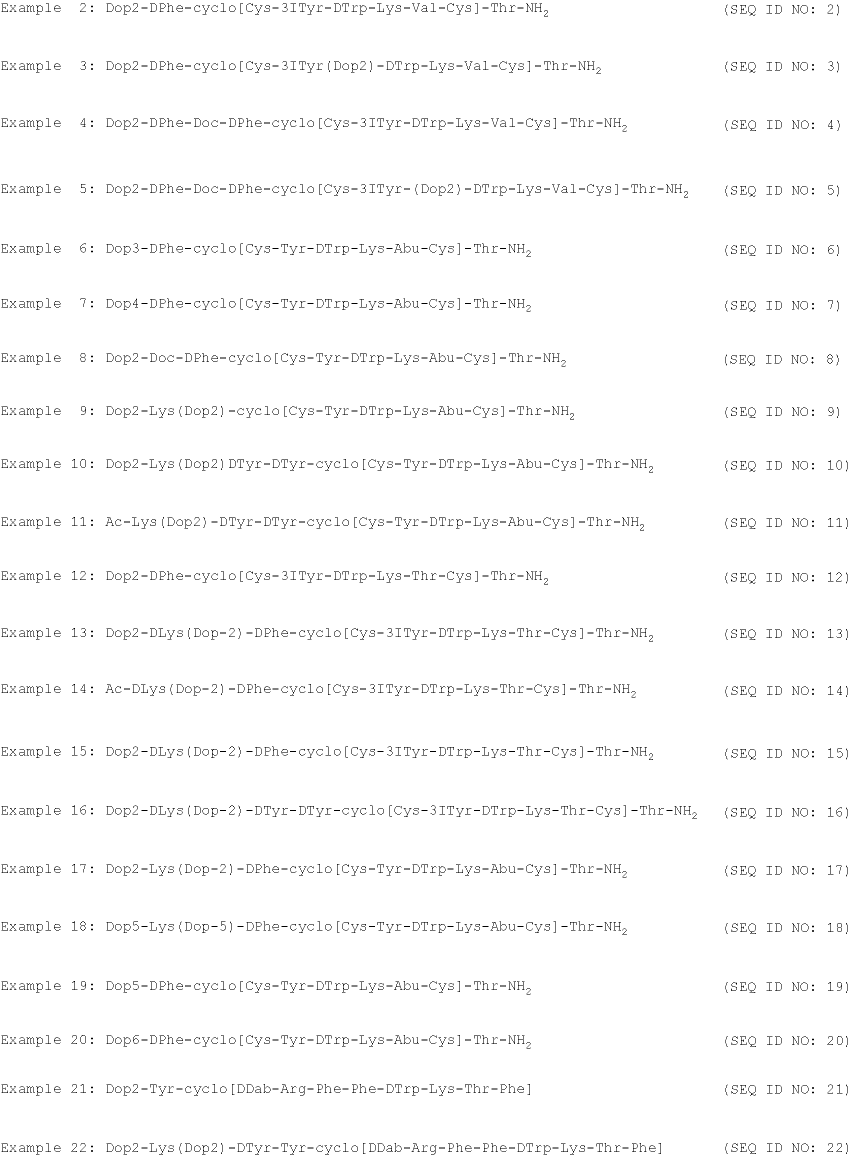

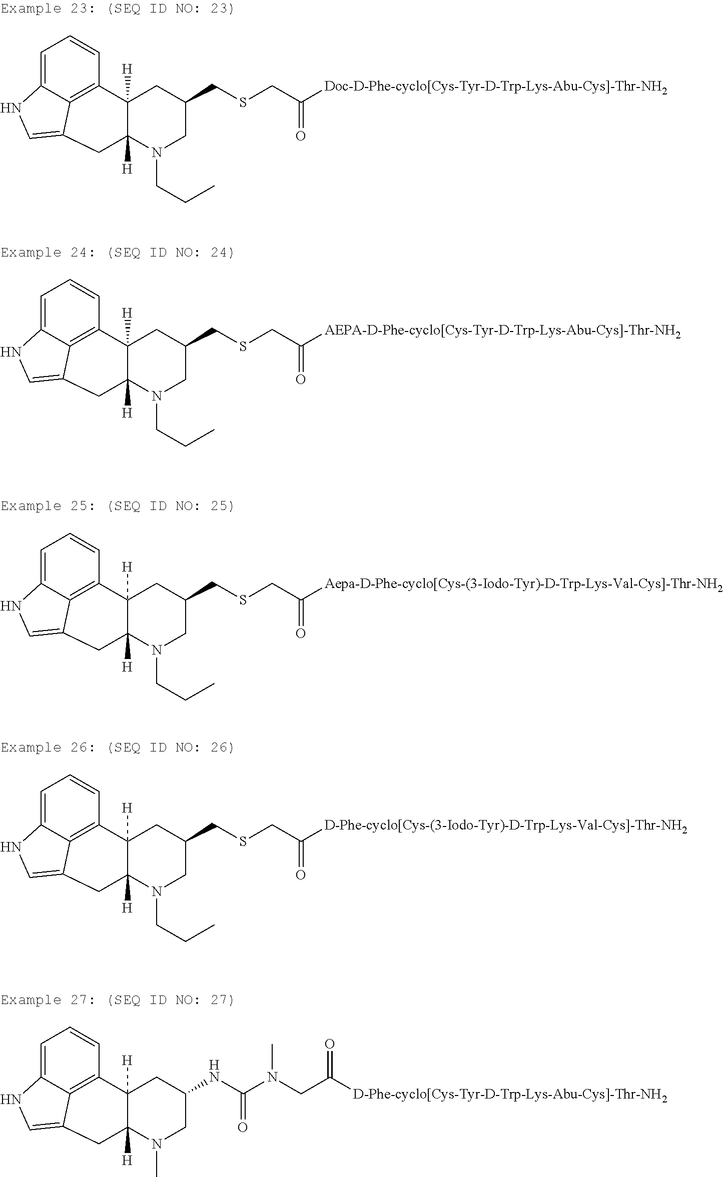

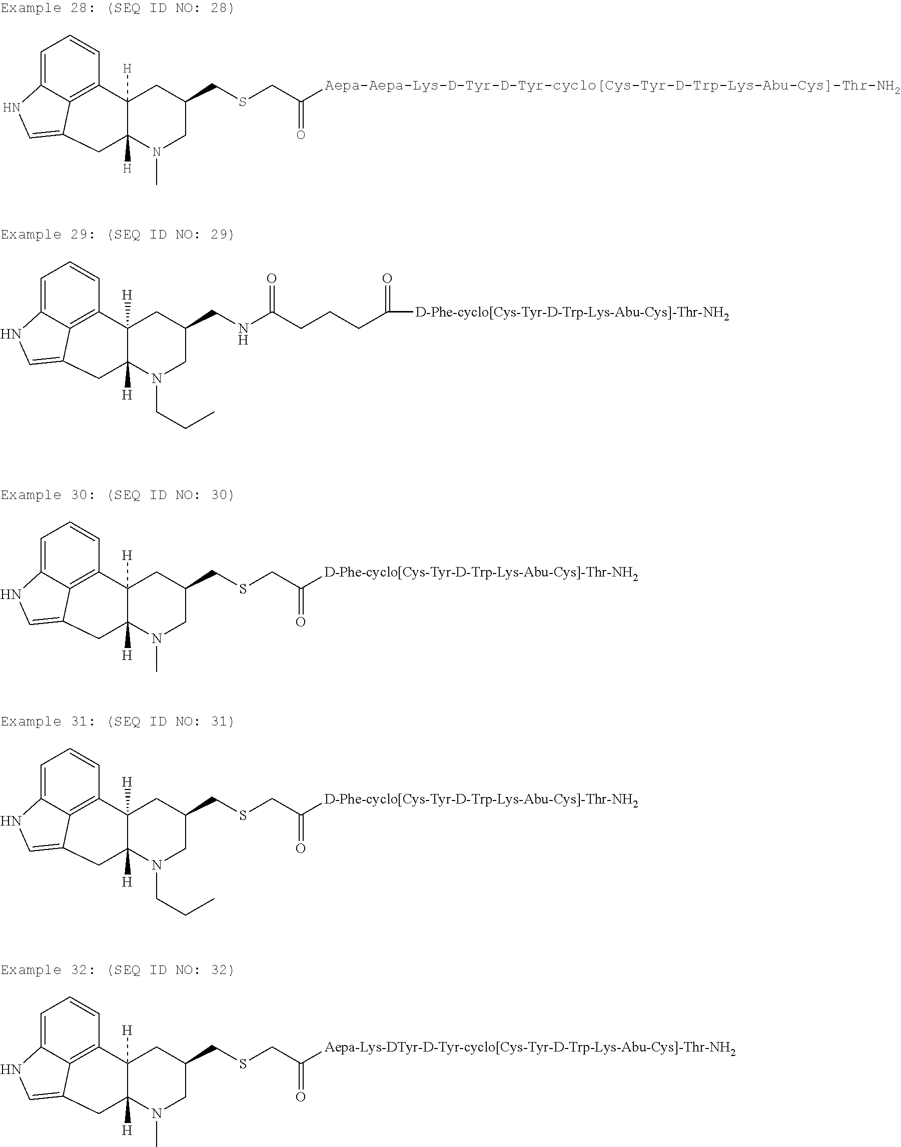

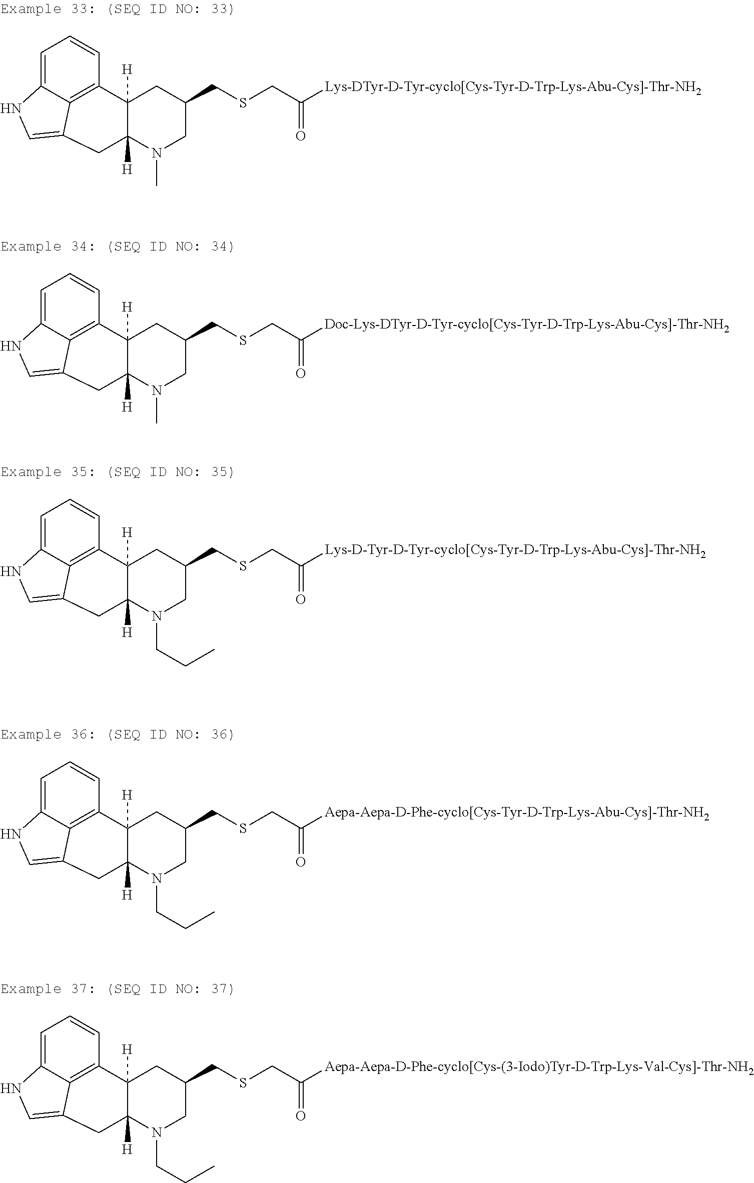

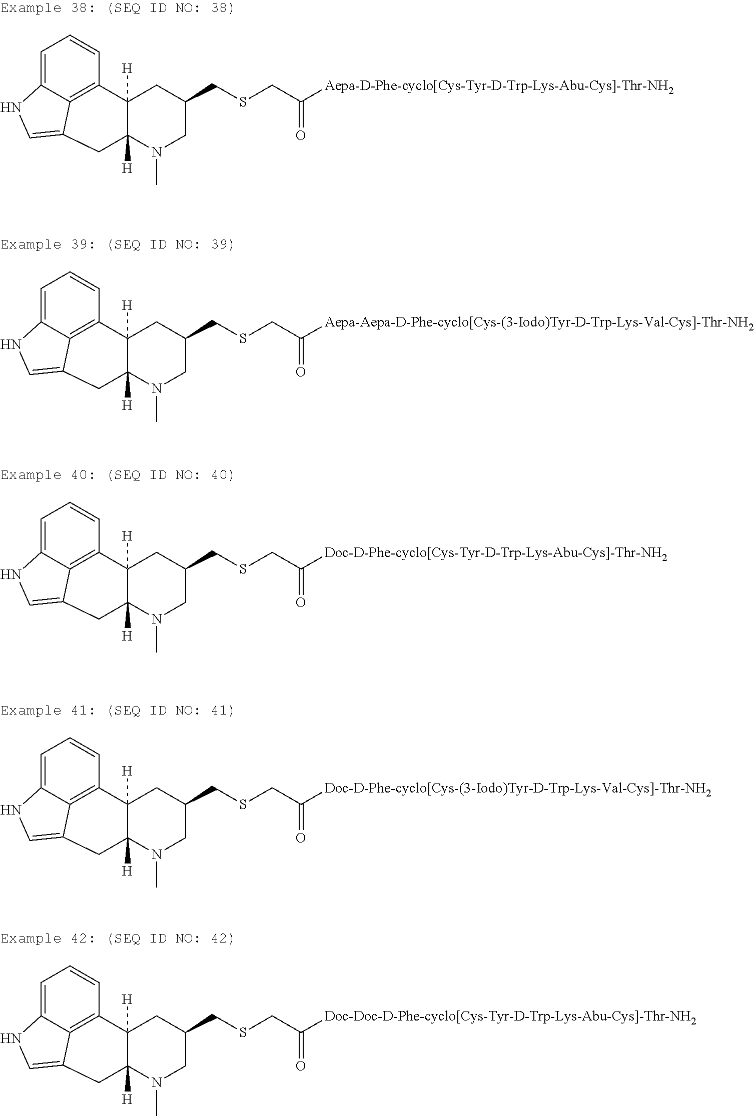

### BRIEF DESCRIPTION OF THE DRAWINGS

FIG. 1 depicts the full time course plasma profiles (median values) obtained after a single subcutaneous administration to Sprague Dawley rats of 20 mg/kg body weight of the following two Example 1 formulations:
200 mg/mL 5% DMA water solution of Example 1; and
200 mg/mL 5% PEG400 water solution of Example 1.

FIG. 2 depicts the estimated percentage of Example 1 remaining at the injection site of Sprague Dawley rats after a single subcutaneous administration of the two test formulations shown in FIG. 1.

FIGS. 3A and 3B depict full time course plasma profiles (median values), on a normal scale and on a logarithmic scale, respectively, obtained after a single subcutaneous administration to Sprague Dawley rats of 1.8 mg/kg body weight of the following Example 1 formulation:
30% (w/v) Example 1 dissolved in 20% of PEG400 water solution.

FIGS. 4A and 4B depict full time course plasma profiles (median values), on a normal scale and on a logarithmic scale, respectively, obtained after a single subcutaneous administration to Sprague Dawley rats of 1.8 mg/kg body weight of the following Example 1 formulation:
15% (w/v) Example 1 in water.

FIGS. 5A and 5B depict full time course plasma profiles (median values), on a normal scale and on a logarithmic scale, respectively, obtained after a single subcutaneous administration to Sprague Dawley rats of 1.8 mg/kg body weight of the following Example 1 formulation:
30% (w/v) Example 1 in water.

### DETAILED DESCRIPTION OF THE INVENTION

By “Dop2” is meant a compound having the structure of:

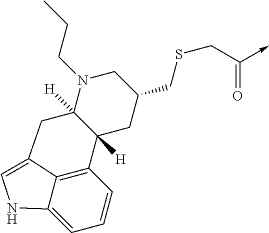

By “Dop3” is meant a compound having the structure of:

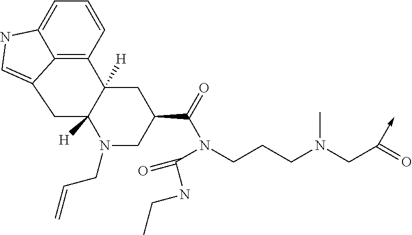

By “Dop4” is meant a compound having the structure of:

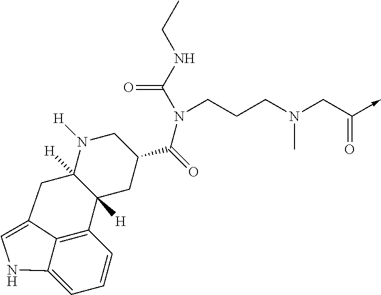

By “Dop5” is meant a compound having the structure of:

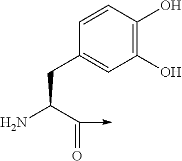

By “Dop6” is meant a compound having the structure of:

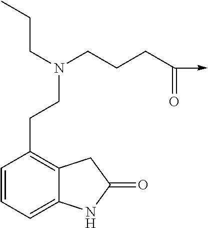

Lys(Dop2) has the structure of:

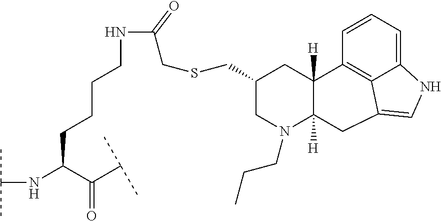

Dop2-Lys(Dop2) has the structure of:

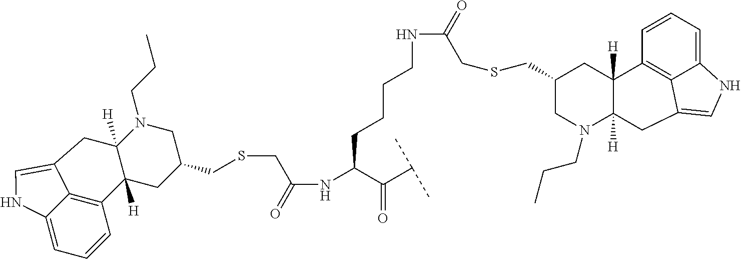

Lys(Dop5) has the structure of:

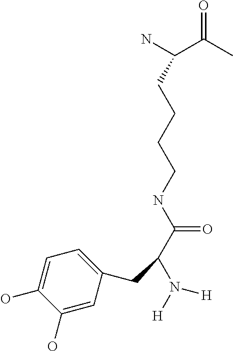

Dop5-Lys(Dop5) has the structure of

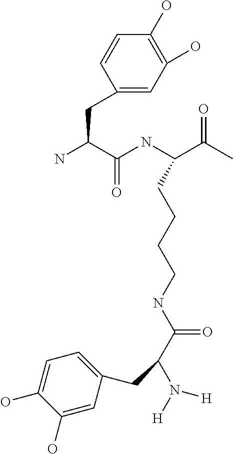

The term “about” as used herein in association with parameters and amounts, means that the parameter or amount is within ±5% of the stated parameter or amount.

By “Aepa” is meant 4-(2-aminoethyl)-1-carboxy methyl-piperazine, represented by the structure:

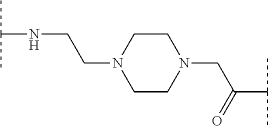

By “Abu” is meant α-aminobutyric acid.

By “Ac” is meant acetyl.

By “BSA” is meant bovine serum albumin.

By “Cys” or “C” is meant cysteine.

By “Dab” is meant 2,4-diaminobutyric acid.

By “DCM” is meant dichloromethane.

By “DIC” is meant N,N-diisopropylcarbodiimide.

By “DIEA” is meant diisopropylethyl amine.

By “DMF” is meant N,N-dimethylformamide.

By “DMA” is meant dimethylacetamide.

By “Fmoc” is meant Fluorenylmethoxycarbonyl.

By “HPLC” is meant high performance liquid chromatography.

By “Lys” or K” is meant lysine.

By “NMP” is meant N-methylpyrrolidone.

By “PBS” is meant phosphate buffered saline, pH 7.4.

By “PEG” is meant polyethylene glycol.

By “PEG300” is meant polyethylene glycol with an average molecular weight of 300. By “PEG400” is meant polyethylene glycol with an average molecular weight of 400. By “PEG1750” is meant polyethylene glycol with an average molecular weight of 1750. By “Thr” or “T” is meant threonine.

By “Trp” or “W” is meant tryptophan.

By “Tyr” or “Y” is meant tyrosine.

By “tBu” is meant tert-butyl.

By “TIS” is meant triisopropylsilane.

By “TFA” is meant trifluoro acetic acid.

By “Val” or “V” is meant valine.

By a “somatostatin receptor agonist” is meant a compound that has a high binding affinity (e.g., Ki of less than 100 nM, or preferably less than 10 nM, or more preferably less than 1 nM) for a somatostatin receptor (e.g., as defined by the receptor binding assay described below), such as any of the different subtypes: e.g., SSTR-1, SSTR-2, SSTR-3, SSTR-4, and SSTR-5, and elicits a somatostatin-like effect; for example, in an assay for the inhibition of cAMP intracellular production.

By a “somatostatin selective agonist” is meant a somatostatin receptor agonist which has a higher binding affinity (i.e., lower Ki) for one somatostatin receptor subtype than for any other somatostatin receptor subtype, such as, for example, a somatostatin SSTR-2 selective agonist.

By a “dopamine receptor agonist” is meant a compound that has a high binding affinity (e.g., Ki of less than 100 nM, or preferably less than 10 nM, or more preferably less than 1 nM) for a dopamine receptor (e.g., as defined by the receptor binding assay described below), such as any of the different subtypes: e.g., D1, D2, D3, D4, and D5 receptors.

Synthesis of Example 1, i.e.,
(SEQ ID NO: 1)
Dop2-DLys(Dop2)-cyclo[Cys-Tyr-DTrp-Lys-Abu-Cys]-
Thr-NH₂

Example 1, i.e., Dop2-DLys (Dop2)-cyclo[Cys-Tyr-DTrp-Lys-Abu-Cys]-Thr-NH₂ (SEQ ID NO:1), was automatically synthesized on an ACT 396 peptide synthesizer (Advanced ChemTech, Louisville, Ky., U.S.A.) using Fmoc chemistry. A Rink Amide 4-methylbenzylhydrylamine (MBHA) resin (Novabiochem., San Diego, Calif., USA) with substitution of 0.66 mmol/g was used (sub: 0.66 mmol/g, 76 mg, 50 mol scale). The Fmoc amino acids used are Fmoc-DLys (Dde)-OH, Fmoc-Cys(Trt)-OH, Fmoc-Tyr(tBu)-OH, Fmoc-DTrp(Boc)-OH, Fmoc-Lys(Boc)-OH, Fmoc-Abu-OH and Fmoc-Thr(tBu)—OH, which were purchased from Novabiochem (San Diego, Calif., USA). The synthesis was carried out on a 50 μmol scale. For each reaction cycle, the ACT 396 peptide synthesizer was programmed to perform: (1) washing with NMP twice; (2) removing Fmoc protecting group with 20% piperidine in NMP for 1×5 min and 1×25 min; (3) washing with NMP twice; and (4) double coupling with 4× fold excess of Fmoc protected amino acid (0.20 mmol), HOBt (0.2 mmol), and DIC (0.2 mmol) in DMF for 1 hour per coupling. The resin was coupled successively according to the sequence.

After the peptide chain was assembled, the Fmoc group was removed and the resin was washed completely with NMP and DCM. The resin was transferred into a reaction vessel on a shaker and treated with 2% hydrazine in DMF for 2×30 minutes to remove Dde protecting group in the side chain of DLys. After washing successively with DMF, MeOH and DCM, the resin was shaken overnight with a solution of Dop2-OH (54 mg, 3.0 eq), bromo-tris-pyrrolidino-phosphonium hexafluorophosphate (PyBrop, 82 mg, 3.4 eq), 1-hydroxy-7-azabenzotriazole (HOAT, 0.4 mg, 3.0 eq), pentafluorophenol (18.4 mg, 4 eq), DMAP (0.25 mL of 0.1 M in DMF, 1.0 eq) and DIEA (53 L, 4 eq).

After washing successively with DMF, MeOH and DCM, the resin was treated with a mixture of TFA (4.75 mL), H₂O (0.4 mL), and TIS (0.425 mL) for 2 hours. The resin was removed by filtration. The filtrate was poured into 70 mL of ether. The precipitate formed was filtered off and washed thoroughly with ether. This crude product was dissolved in 5 mL of aqueous acetic acid solution (water/acetic acid=1:1). The solution was then diluted with 50 mL of H₂O and 20 mL of acetonitrile, to which was added iodine in methanol until the solution sustained yellow. The solution was stirred slowly for 1 hour and the reaction was terminated by adding aqueous Na₂S₂O₃ solution. The crude product was purified on reverse-phase preparative HPLC using a column of C18 Dynamax-100A⁰ (4×43 cm, Varian, Walnut Creek, Calif., USA). The column was eluted with a liner gradient from 90% A and 10% B to 60% A and 40% B in an hour where A was 0.1% TFA in water and B was 0.1% TFA in acetonitrile. Fractions containing a major component by ultraviolet absorption were pooled and lyophilized. The purity was 99.99% based on an analytical HPLC analysis. Electro-spray ionization mass spectrometry (ES-MS) analysis gave the molecular weight at 1693.60 (in agreement with the calculated molecular weight of 1694.23).

The other exemplified somatostatin-dopamine conjugates were synthesized substantially according to the procedure described for the synthesis of Example 1. Physical data for the exemplified somatostatin-dopamine conjugates are given in Table 1.

TABLE 1

| Example | Mol. Wt. | Mol. Wt. | Purity |
|---|---|---|---|
| Number | Expected | (ES-MS) | (HPLC) |
| 1 | 1694.23 | 1693.60 | 99.99% |
| 2 | 1512.66 | 1512.67 | 89.00% |
| 3 | 1853.15 | 1853.70 | 94.90% |
| 4 | 1804.99 | 1804.60 | 90.90% |
| 5 | 2145.48 | 2145.90 | 95.40% |
| 6 | 1509.86 | 1512.20 | 95.00% |
| 7 | 1469.79 | 1469.70 | 84.00% |
| 8 | 1517.90 | 1517.70 | 99.99% |
| 9 | 1694.23 | 1693.60 | 91.70% |
| 10 | 2020.58 | 2020.90 | 93.90% |
| 11 | 1722.12 | 1721.20 | 98.50% |
| 12 | 1514.63 | 1514.50 | 99.99% |
| 13 | 1983.29 | 1982.60 | 95.70% |
| 14 | 1684.84 | 1684.50 | 91.60% |
| 15 | 1983.29 | 1983.10 | 99.99% |
| 16 | 2162.47 | 2162.40 | 99.99% |
| 17 | 1841.40 | 1840.80 | 96.70% |
| 18 | 1518.77 | 1518.40 | 99.99% |
| 19 | 1211.43 | 1211.30 | 99.99% |
| 20 | 1370.66 | 1370.58 | 92.00% |
| 21 | 1616.99 | 1616.80 | 95.00% |
| 22 | 2248.83 | 2248.40 | 98.00% |
| 23 | 1517.90 | 1517.70 | 99.99% |
| 24 | 1541.97 | N/A | 97.80% |
| 25 | 1681.89 | N/A | 97.20% |
| 26 | 1512.66 | 1512.67 | 89.00% |
| 27 | 1370.66 | 1370.58 | 92.00% |
| 28 | 1990.49 | N/A | 99.00% |
| 29 | N/A | N/A | N/A |
| 30 | 1344.69 | 1343.80 | 98.20% |
| 31 | 1372.74 | 1371.50 | 95.00% |
| 32 | 1821.26 | N/A | 96.80% |
| 33 | 1652.03 | 1651.90 | 97.90% |
| 34 | 1797.19 | 1796.10 | 99.20% |
| 35 | 1680.09 | N/A | 97.40% |
| 36 | 1711.19 | N/A | 99.90% |
| 37 | 1851.11 | N/A | 99.00% |
| 38 | 1513.91 | N/A | 98.30% |
| 39 | 1823.06 | N/A | 85.70% |
| 40 | 1489.84 | 1489.70 | 98.90% |
| 41 | 1956.34 | 1956.37 | 96.40% |
| 42 | 1635.00 | 1634.70 | 97.00% |

Somatostatin Receptor Specificity and Selectivity Assay

Specificity and selectivity of the somatostatin analogues used to synthesize the somatostatin-dopamine chimers were determined by a radioligand binding assay on CHO-K1 cells stably transfected with each of the SSTR subtypes, as follows. Somatostatin analogs are also described in U.S. Patent Application Publication No. 02210006790. The complete coding sequences of genomic fragments of the SSTR 1 (e.g., Genbank accession No. M81829), SSTR 2 (e.g., Genbank accession No. M81830), SSTR 3 (e.g., Genbank accession No. L07062), and SSTR 4 (e.g., Genbank accession No. AL049651) genes and a cDNA clone for SSTR 5 (e.g., Genbank accession No. D16827) was subcloned into the mammalian expression vector pCMV (Life Technologies, Milano, Italy). Other SSTR sequences are known to the skilled artisan. Clonal cell lines stably expressing SSTR's 1-5 were obtained by transfection into CHO-K₁ cells (ATCC, Manassas, Va., USA) using the calcium phosphate co-precipitation method (Davis L, et al., 1994 In: Basic methods in Molecular Biology, 2nd edition, Appleton & Lange, Norwalk, Conn., USA: 611-646). The plasmid pRSV-neo (ATCC) was included as a selectable marker. Clonal cell lines were selected in RPMI 1640 media containing 0.5 mg/mL of G418 (Life Technologies, Milano, Italy), ring cloned, and expanded into culture.

Membranes for in vitro receptor binding assays were obtained by homogenizing the CHO-K1 cells expressing the SSTR's subtypes in ice-cold 50 mM Tris-HCl and centrifuging twice at 39,000 g (10 min), with an intermediate resuspension in fresh buffer. The final pellets were resuspended in 10 mM Tris-HCl for assay.

For the SSTR 1, 3, 4, and 5 assays, aliquots of the membrane preparations were incubated 90 minutes at 25° C. with 0.05 nM [¹²⁵I-Tyr11]SS-14 in 50 mM HEPES (pH 7.4) containing 10 mg/mL BSA, 5 mM MgCl₂, 200 KIU/mL Trasylol, 0.02 mg/mL bacitracin, and 0.02 mg/mL phenylmethylsuphonyl fluoride. The final assay volume was 0.3 mL.

For the SSTR 2 assay, 0.05 nM [¹²⁵I]MK-678 was employed as the radioligand and the incubation time was 90 minutes at 25° C. The incubations were terminated by rapid filtration through GF/C glass microfiber filters (Whatman Co.) (pre-soaked in 0.3% polyethylenimine) using a BRANDEL filtration manifold. Each tube and filter was washed three times with 5 mL aliquots of ice-cold buffer. Specific binding was defined as the total radioligand bound minus that bound in the presence of 1000 nM SS-14 for SSTR 1, 3, 4, and 5, or 1000 nM MK-678 for SSTR2.

Dopamine Receptor Specificity and Selectivity Assay

Specificity and selectivity for the dopamine-2 receptor of the dopamine analogues used to synthesize the somatostatin-dopamine chimers may be determined by a radioligand binding assay as follows.

Crude membranes were prepared by homogenization of frozen rat corpus striatum (Zivic Laboratories, Pittsburgh, Pa., USA) in 20 mL of ice-cold 50 mM Tris-HCl with a Brinkman Polytron cell disrupter (setting 6, 15 sec). Buffer was added to obtain a final volume of 40 mL, and the homogenate was centrifuged in a Sorval SS-34 rotor at 39,000 g for 10 minutes at 0-4° C. The resulting supernatant was decanted and discarded. The pellet was rehomogenized in ice-cold buffer, pre-incubated at 37° C. for 10 min, diluted, and centrifuged as before. The final pellet was resuspended in buffer and held on ice for the receptor binding assay.

For assay, aliquots of the washed membrane preparations and test compounds were incubated for 15 minutes (37° C.) with 0.25 nM [³HI]spiperone (16.5 Ci.mmol, New England Nuclear, Boston, Mass., USA) in 50 mM Tris HCl, 120 mM NaCl, 5 mM KCl, 2 mM CaCl₂, 1 mM MgCl₂. The final assay volume was 1.0 mL. The incubations were terminated by rapid filtration through GF/B glass fibre filters using a Brandel filtration manifold. Each tube and filter was then washed three times with 5-mL aliquots of ice-cold buffer. Specific binding was defined as the total radioligand bound minus that bound in the presence of 1000 nM (+) butaclamol.

Using the discussed assays, the inhibition constants (Ki) for the five human somatostatin receptors (hSSTR1-hSSTR5) and the dopamine-2 receptor (hUTII and hDA2) were measured for the exemplified somatostatin-dopamine conjugates, as follows:

TABLE 2

| Ex- | | | | | | | |
|---|---|---|---|---|---|---|---|
| am- | | | | | | | |
| ple | | | | | | | |
| Num- | hsst1 | hsst2 | hsst3 | hsst4 | hsst5 | hUTII | hDA2 |
| ber | Ki (nM) | Ki (nM) | Ki (nM) | Ki (nM) | Ki (nM) | Ki (nM) | Ki (nM) |
| 1 | 843.00 | 0.03 | 160.00 | 1000.00 | 41.51 | 508.50 | 15.85 |
| 2 | 730.64 | 0.40 | 135.00 | 1000.00 | 7.02 | 53.19 | 34.05 |
| 3 | 1000.00 | 0.37 | 235.00 | 1000.00 | 13.65 | 73.50 | 16.71 |
| 4 | 1000.00 | 0.84 | 397.00 | 1000.00 | 21.17 | 83.72 | 29.56 |
| 5 | 1000.00 | 1.65 | 1054.00 | 1000.00 | 27.56 | 104.74 | 15.48 |
| 6 | 509.00 | 0.51 | 798.00 | 1000.00 | 56.46 | 676.74 | 64.97 |
| 7 | 345.00 | 0.19 | 267.00 | 1000.00 | 28.58 | 695.33 | 192.96 |
| 8 | 1548.00 | 0.11 | 126.00 | 1000.00 | 24.46 | 166.77 | 86.03 |
| 9 | 273.00 | 0.54 | 536.00 | 1000.00 | 99.52 | 634.50 | 8.30 |
| 10 | 549.00 | 0.15 | 324.00 | 1000.00 | 26.54 | 177.20 | 8.22 |
| 11 | 437.00 | 0.04 | 162.00 | 1000.00 | 8.91 | 64.41 | 119.12 |
| 12 | 602.00 | 0.06 | 51.50 | 1000.00 | 4.10 | 676.00 | 25.21 |
| 13 | 907.00 | 0.12 | 196.00 | 1000.00 | 10.71 | 961.00 | 15.17 |
| 14 | 1338.00 | 0.07 | 70.30 | 1000.00 | 2.68 | 1509.50 | 44.33 |
| 15 | N/A | 1.21 | 196.00 | 1000.00 | 6.29 | 300.50 | 18.34 |
| 16 | N/A | 0.16 | 76.40 | 1000.00 | 7.43 | 549.39 | 8.56 |
| 17 | N/A | 0.18 | 106.00 | 1000.00 | 54.04 | 495.93 | 17.58 |
| 18 | N/A | 0.36 | 167.00 | 1000.00 | 31.99 | 1000.00 | 3000.00 |
| 19 | N/A | 0.41 | 146.00 | 1000.00 | 19.70 | 2250.58 | 3000.00 |
| 20 | N/A | 0.02 | 140.00 | 1000.00 | 22.77 | 1278.70 | 95.15 |
| 21 | N/A | N/A | N/A | N/A | 0.00 | 1061.00 | N/A |
| 22 | N/A | N/A | N/A | N/A | 0.00 | 2483.00 | N/A |
| 23 | 1548.00 | 0.11 | 126.00 | 1000.00 | 24.46 | 166.77 | 86.03 |
| 24 | 1000.00 | 0.37 | 154.40 | 1000.00 | 24.16 | 1511.00 | 142.82 |
| 25 | 1000.00 | 1.09 | 423.00 | 1000.00 | 14.30 | 233.33 | 345.00 |
| 26 | 730.64 | 0.40 | 135.00 | 1000.00 | 7.02 | 53.19 | 34.05 |
| 27 | N/A | 0.02 | 140.00 | 1000.00 | 22.77 | 1278.70 | 95.15 |
| 28 | 660.71 | 0.11 | 156.00 | 1000.00 | 5.13 | 564.30 | 506.33 |
| 29 | N/A | 0.14 | N/A | N/A | N/A | N/A | 226.50 |
| 30 | 688.90 | 0.15 | 49.51 | 1000.00 | 15.72 | 619.39 | 23.74 |
| 31 | 690.67 | 0.10 | 84.60 | 1000.00 | 22.65 | 497.50 | 25.47 |
| 32 | N/A | 0.22 | 45.57 | 1000.00 | 13.78 | 266.50 | 136.18 |
| 33 | N/A | 0.20 | 65.11 | 1000.00 | 9.82 | 37.68 | 205.50 |
| 34 | N/A | 0.23 | 48.45 | 1000.00 | 8.60 | 211.00 | 114.42 |
| 35 | N/A | 0.27 | 109.26 | 1000.00 | 13.64 | 134.00 | 55.83 |
| 36 | N/A | 1.67 | 145.06 | 1000.00 | 53.48 | 3085.15 | 328.00 |
| 37 | N/A | 1.26 | 373.55 | 1000.00 | 10.11 | 420.52 | 126.50 |
| 38 | N/A | 0.35 | 61.85 | 1000.00 | 28.06 | 1190.32 | 411.00 |
| 39 | N/A | 0.42 | 136.30 | 1000 | 5.45 | 241.41 | 310.00 |
| 40 | 1000.00 | 0.32 | 70.32 | 1000.00 | 16.99 | 222.69 | 255.00 |
| 41 | 415.53 | 0.70 | 165.13 | 1000.00 | 6.93 | 26.97 | 217.00 |
| 42 | 1000.00 | 0.34 | 120.05 | 1000.00 | 20.63 | 509.98 | 277.67 |

Inhibition of cAMP Intracellular Production

Somatostatin (sst) and dopamine (D₂) receptor subtypes are co-expressed in various neuro-endocrine tumors and may show functional synergism. Novel somatostatin-dopamine chimeric molecules as disclosed herein, such as Example 1, that bind to both receptor subtypes have displayed superagonistic properties in some earlier preclinical studies. This may be either due to the induction of heterodimerization of their target receptors at the plasma membrane or to enhanced activation of the individual receptors of these compounds.

A cAMP Responsive Element-Luciferase reporter gene assay in HEK-293 cells was used in this assay, wherein said HEK-293 cells were transiently transfected with D₂ and/or sst₂ cDNA. In D₂-monotransfected cells, the IC₅₀ value of cAMP inhibition of Example 1 was 0.02 nM. In sst₂-monotransfected cells, the IC₅₀ value of cAMP inhibition of Example 1 was 0.04 nM. In sst₂-D₂ co-transfected cells, the IC₅₀ value of cAMP inhibition of Example 1 was 0.02 nM.

It can be concluded that in this cell model, Example 1 mediates most of its superpotent effects through high-affinity binding and activation of D₂ receptors. The superior activation of D2 receptors in combination with a high potency activation of sst2 receptors could explain the superagonistic effects that have been observed with this compound in several preclinical studies.

Determination of Solubility of Example 1 at Various Concentrations of DMA and PEG400

A compound that may advantageously be used to practice the invention can be tested to determine its solubility at different DMA and PEG400 concentrations using the following procedure.

Solvents used are:

5%, 10%, 20%, 30%, 40% DMA, 5%, 10%, 20%, 30%, 40% PEG400 in water; and

5%, 10%, 20%, 30%, 40% DMA, 5%, 10%, 20%, 30%, 40% PEG400 in PBS.

To about 1 mg of Example 1 were added increasing volumes of the above solvents or buffers. When a soluble volume was reached, the concentration was calculated by weight/volume. When Example 1 was not soluble, the solution was centrifuged and the supernatant was analyzed by HPLC to determine the concentrations. The determined concentration is treated as the solubility of Example 1 in that solvent or buffer.

The solution pHs were checked. They were about pH 7. No further adjustment was done.

The solubility of Example 1 in water and PBS are very different. Example 1 is much more soluble in water based solvents than in PBS based solvents. Therefore, both water and PBS based solvents were used in this study. The results are listed in the following tables.

TABLE 3

| | Solubility |
|---|---|
| Water pH 7 | (mg/mL) |
| 0% DMA | 0.86* |
| 5% DMA | >100 |
| 10% DMA | >100 |
| 20% DMA | >100 |
| 30% DMA | >100 |
| 40% DMA | >100 |
| 5% PEG400 | >50 |
| 10% PEG400 | >50 |
| 20% PEG400 | >50 |
| 30% PEG400 | >50 |
| 40% PEG400 | >50 |

*by HPLC

TABLE 4

| | Solubility |
|---|---|
| PBS pH 7 | (mg/mL) |
| 0% DMA | 0.04* |
| 5% DMA | 0.13* |
| 10% DMA | 0.48* |
| 20% DMA | 0.78* |
| 30% DMA | 4.20* |
| 40% DMA | >50 |
| 5% PEG400 | 0.05* |
| 10% PEG400 | 0.33* |
| 20% PEG400 | 0.75* |
| 30% PEG400 | 2.98* |
| 40% PEG400 | 3.50* |

*by HPLC

Pharmacokinetic Studies of Example 1 Formulations

Five different formulations of Example 1 (“Formulations 1-5”) were prepared by using the following procedures:
(1) Example 1 was dissolved in 5% DMA water solution at the concentration of 200 mg/mL.
(2) Example 1 was dissolved in 5% PEG400 water solution at the concentration of 200 mg/mL.
(3) Example 1 was dissolved in 20% PEG400 water solution at the concentration of 30% (w/v).
(4) Example 1 was dissolved in water at the concentration of 15% (w/v).
(5) Example 1 was dissolved in water at the concentration of 30% (w/v).

Dosing and Blood Sample Collection

For Formulations (1) and (2), Sprague Dawley rats were dosed at 20 mg/kg body weight subcutaneously with these formulations of Example 1. Blood samples were collected at 1, 2, 4, 8, 24 hours, and 2, 3, 4, 7 days. Plasma was collected from the blood by centrifugation and stored at −80° C. Tissues at the injection site were also collected, homogenized with 5× methanol, and stored at −80° C.

For Formulations (3), (4) and (5), Sprague Dawley rats were dosed at 1.8 mg/kg body weight subcutaneously with these formulations of Example 1. Blood samples were collected at 5, 10, 15, 30 minutes, 1, 2, 4, 8 hours, and 1, 2, 3, 4, 7, 14, 21, 28, 35, 42 days. Plasma was collected from the blood by centrifugation and stored at −80° C. Tissue at the injection site were also collected, homogenized with 5× methanol, and stored at −80° C.

Sample Preparation

Plasma (200 μL) was acidified with 10 μL formic acid and precipitated with 600 μL acetonitrile. The supernatant was collected by centrifugation and concentrated to dryness under vacuum. The residues were dissolved in 150 μL 30% acetonitrile in water and centrifuged. 50 μL of the supernatant was injected for LC-MS/MS analysis.

Tissue methanol extract (10 μL) was diluted to 1 mL 30% acetonitrile in water and 50 μL was injected for LC-MS/MS analysis.

LC-MS/MS Analysis

LC-MS/MS analysis was done with an API4000 mass spectrometer system equipped with a Turbo Ionspray probe. The MRM mode of molecular ion detection was used with the ion pair of 565.6 and 159.1.

HPLC separation was performed with a Luna C8(2) 2×30 mm 3μ column run from 10% B to 90% B in 10 minutes at a flow rate of 0.30 mL/minute. Buffer A is 1% formic acid in water and buffer B is 1% formic acid in acetonitrile.

LOQ was 0.2 ng/mL.

Results and Summary

Formulations (1) and (2)

The plasma concentrations of Example 1 were calculated with its standard calibration plot. 1.5 mg/mL Example 1 (20 mg/kg of 300 g rat in 4 mL methanol extract) was used as the 100% to calculate the percentages left at the injection sites.

TABLE 5
Example 1 plasma concentrations and percentages left at the injection
sites of Example 1, dosed with Formulations (1) and (2)

| | Plasma concentration | Plasma concentration | % left at injection | % left at injection |
|---|---|---|---|---|
| | (ng/mL) of Example | (ng/mL) of Example | site of Example | site of Example |
| | 1, dosed with | 1, dosed with | 1, dosed with | 1, dosed with |
| Time, h | Formulation 1 | Formulation 2 | Formulation 1 | Formulation 12 |
| 1 | 1.7 | 0.4 | Not collected | Not collected |
| 2 | 4.8 | 4.4 | Not collected | Not collected |
| 4 | 6.1 | 2.5 | Not collected | Not collected |
| 8 | 4.7 | 3.5 | Not collected | Not collected |
| 24 | 2.6 | 4.2 | 102 | 13.8 |
| 48 | 1.9 | 1.6 | 67.5 | 69 |
| 72 | 2.3 | 0.5 | 18.1 | 56.8 |
| 96 | 1.8 | 0.4 | 37.6 | 7.4 |
| 168 | 0.2 | 0.3 | 58.8 | 78.9 |

Full time course plots of the pharmacokinetic profiles of Formulations (1) and (2) are shown in FIG. 1.

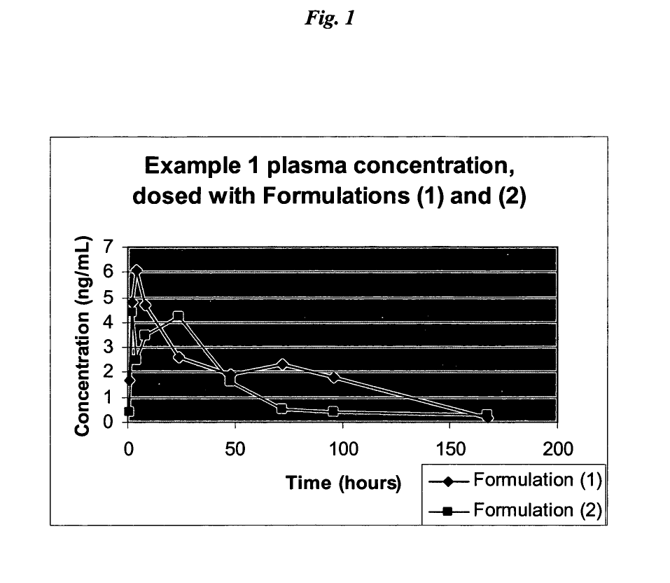

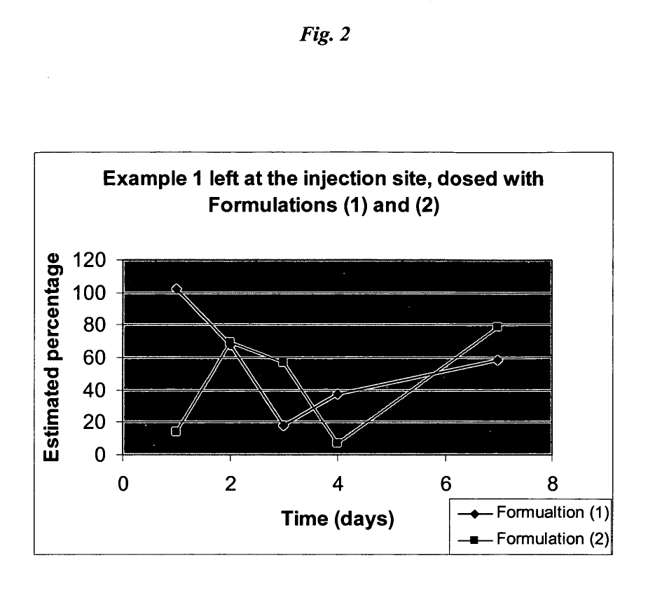

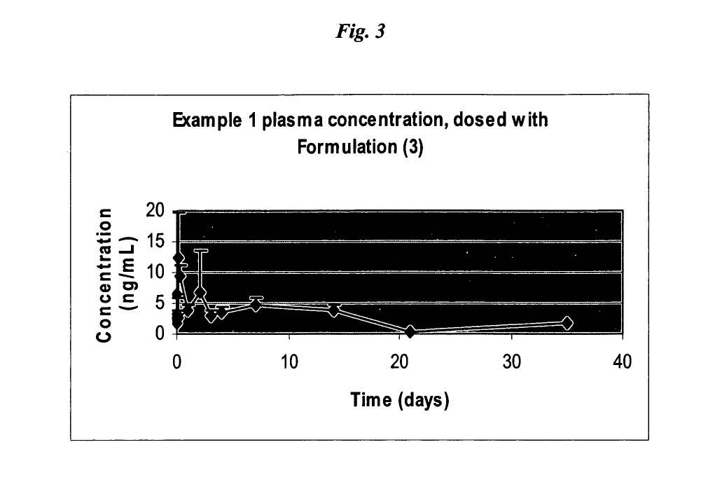

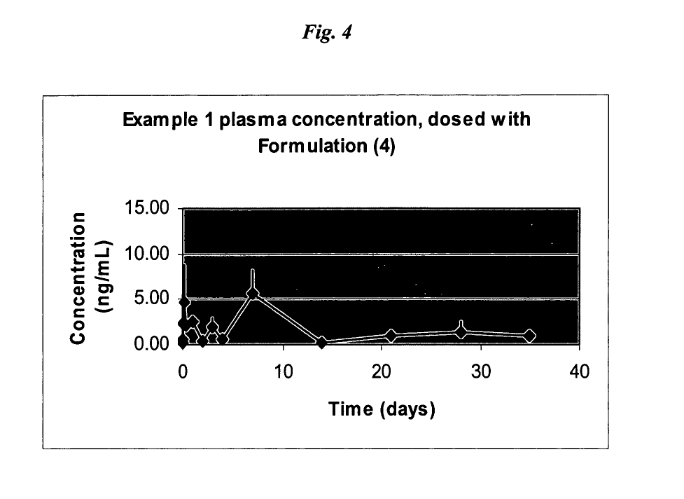

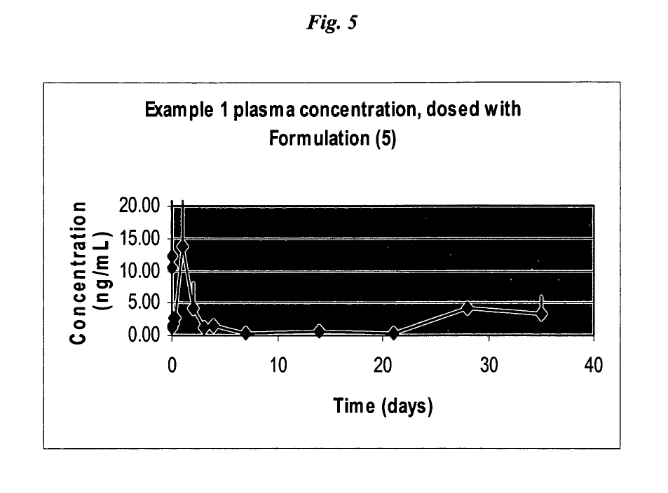

The tissue accumulation profile of Example 1 at the injection site, dosed with Formulations (1) and (2) is shown in FIG. 2.

TABLE 6
Example 1 plasma concentrations dosed
with Formulations (3), (4) and (5)

| | Plasma | Plasma | Plasma |
|---|---|---|---|
| | concentration | concentration | concentration |
| | (ng/mL) of | (ng/mL) of | (ng/mL) of |
| | Example 1, | Example 1, | Example 1, |
| | dosed with | dosed with | dosed with |
| Time | Formulation 3 | Formulation 4 | Formulation 5 |

| | | | | |
|---|---|---|---|---|
| 5 | min | 2.73 | 0.10 | 10.43 |
| 10 | min | 1.88 | 2.33 | 1.05 |
| 15 | min | 3.32 | 0.16 | 12.10 |
| 30 | min | 2.29 | 0.29 | 1.49 |
| 1 | hour | 6.54 | 2.40 | 1.59 |
| 2 | hour | 2.53 | 0.64 | 1.77 |
| 4 | hour | 12.50 | 4.76 | 2.77 |
| 8 | hour | 9.31 | 2.11 | 2.71 |
| 1 | day | 3.94 | 2.54 | 13.75 |
| 2 | day | 6.81 | 0.43 | 4.06 |
| 3 | day | 2.86 | 1.95 | 1.28 |
| 4 | day | 3.56 | 0.62 | 1.62 |
| 7 | day | 4.64 | 5.68 | 0.34 |
| 14 | day | 3.87 | 0.10 | 0.62 |
| 21 | day | 0.40 | 1.05 | 0.36 |
| 28 | day | no peak | 1.46 | 4.13 |
| 35 | day | 1.69 | 0.93 | 3.26 |

Full time course plots of the pharmacokinetic profiles of Formulation (3) are shown in FIG. 3A on a normal scale and FIG. 3B on a logarithmic scale.

Full time course plots of the pharmacokinetic profiles of Formulation (4) are shown in FIG. 4A on a normal scale and FIG. 4B on a logarithmic scale.

Full time course plots of the pharmacokinetic profiles of Formulation (5) are shown in FIG. 5A on a normal scale and FIG. 5B on a logarithmic scale.

TABLE 7
PK parameters

| | Example 1 | Example 1 | Example 1 | Example 1 | Example 1 |
|---|---|---|---|---|---|
| | dosed with | dosed with | dosed with | dosed with | dosed with |
| | Formulation 1 | Formulation 2 | Formulation 3 | Formulation 4 | Formulation 5 |
| Tₘₐₓ, h | 4.0 | 2.0 | 4 | 4 | 4 |
| Cₘₐₓ, ng/mL | 6.1 | 4.4 | 12.5 | 4.76 | 2.77 |
| AUC, ng-hr/mL | 332 | 231 | 2213 | 1374 | 1695 |

The results indicate that the formulations of Example 1 according to the present invention as described herein provide for acceptable sustained release formulations with reduced initial plasma concentrations, which may reduce or eliminate unwanted side-effects. The data also indicate that, after the subcutaneous injection, the body fluid is able to dilute the organic contents of Formulations (1), (2) and (3), and result in the rapid precipitation of Example 1.

Additional embodiments of the present invention will be apparent from the foregoing disclosure and are intended to be encompassed by the invention as described fully herein and defined in the following claims.

1-46. (canceled)

47. A pharmaceutical composition of a clear aqueous solution, or a gel or a semi-solid, comprising a somatostatin-dopamine conjugate, or a pharmaceutically acceptable salt thereof, in which the somatostatin-dopamine conjugate forms a precipitate, or a depot or an in situ deposit, after subcutaneous or intramuscular administration to a subject.

48. The pharmaceutical composition according to claim 47, wherein said somatostatin-dopamine conjugate is: 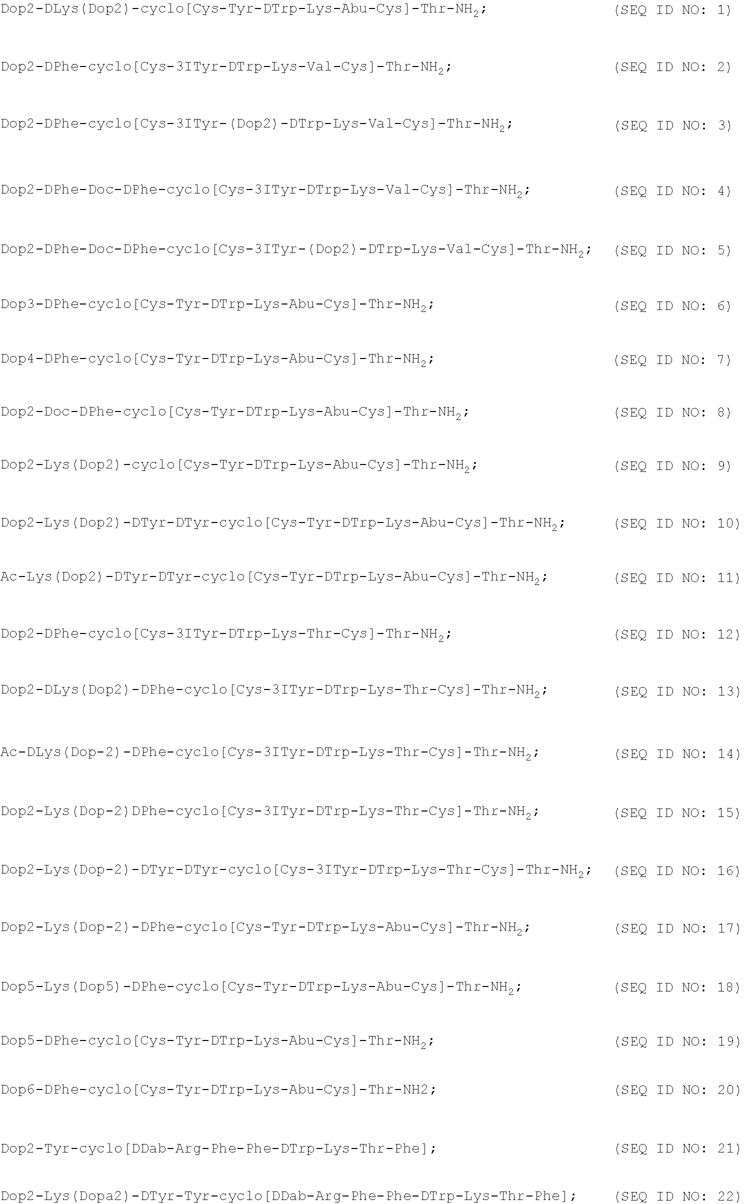 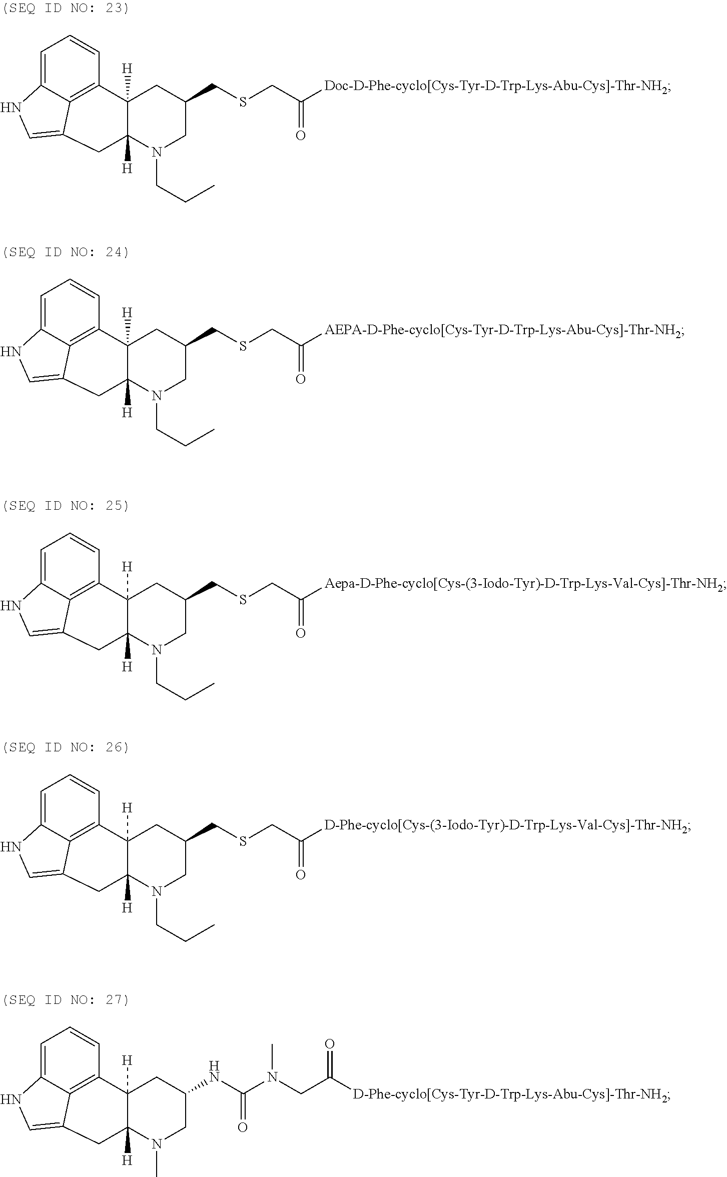 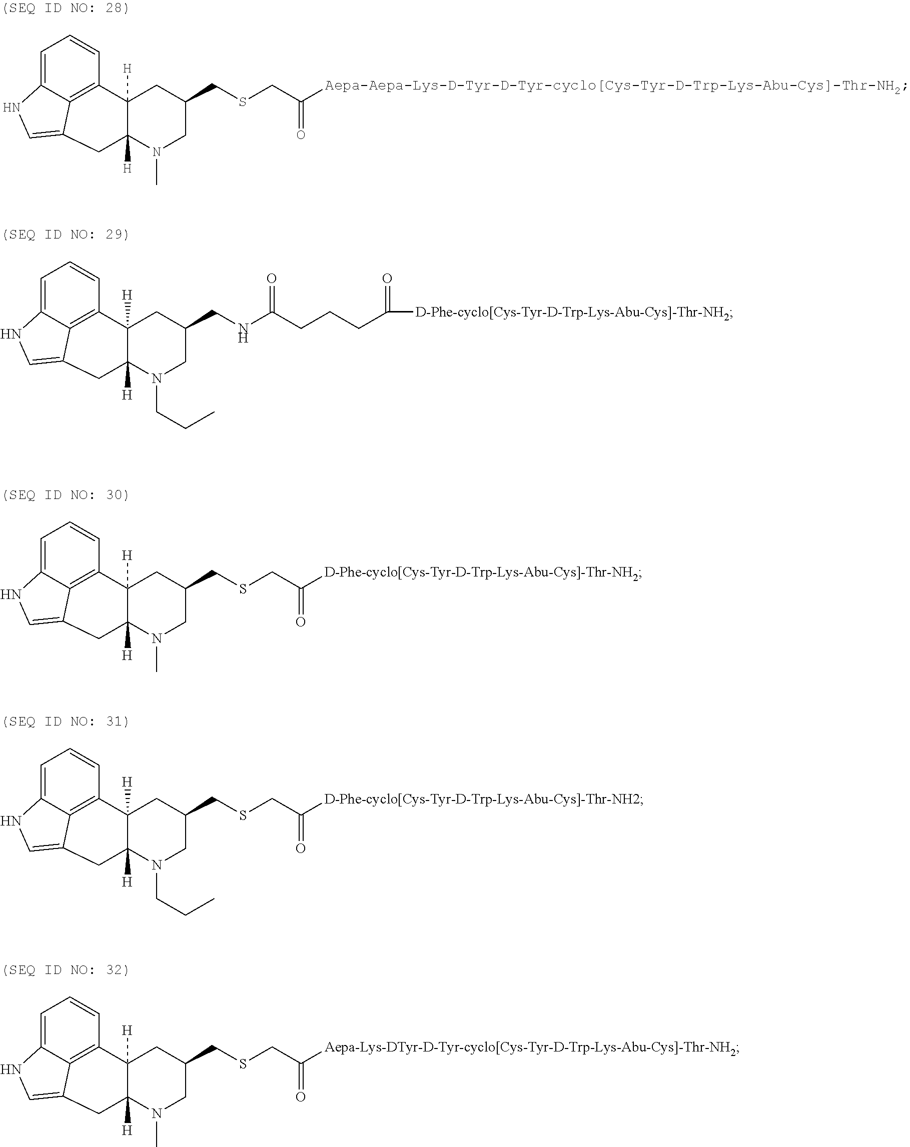 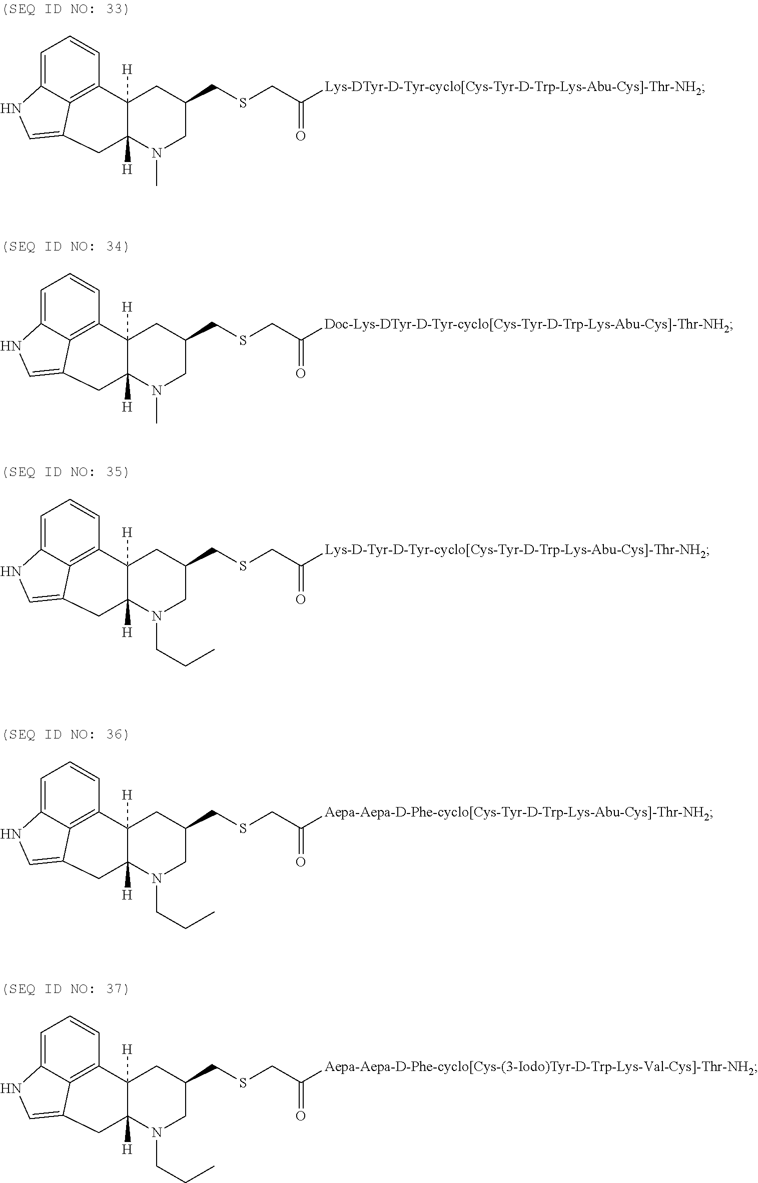 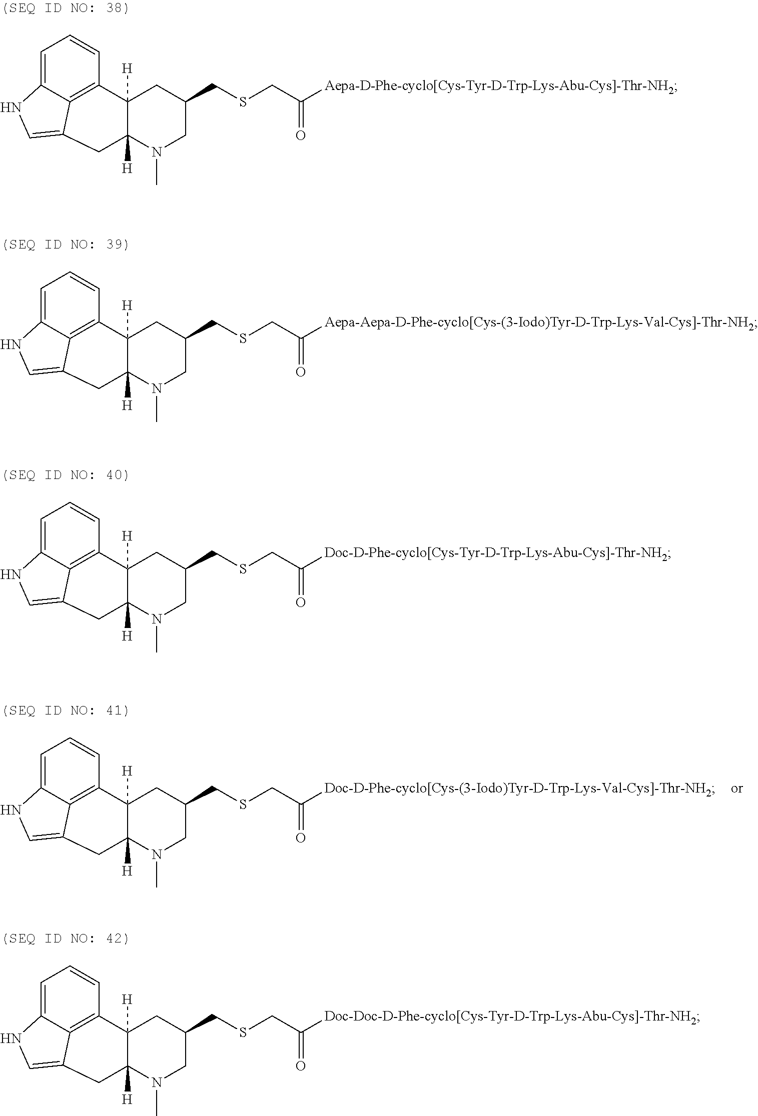
or a pharmaceutically acceptable salt thereof.

49. The pharmaceutical composition according to claim 48, wherein said somatostatin-dopamine conjugate is Dop2-DLys(Dop2)-cyclo[Cys-Tyr-DTrp-Lys-Abu-Cys]-Thr-NH₂ (SEQ ID NO:1).

50. The pharmaceutical composition according to claim 1, further comprising an organic component, wherein said organic component increases solubility of the somatostatin-dopamine conjugate in an aqueous solution or decreases viscosity of a gel or a semi-solid; and wherein

51. The pharmaceutical composition according to claim 50, wherein said organic component is selected from the group consisting of an organic polymer, an organic solvent, an alcohol, a sugar, a cyclodextrin, a phospholipid, a water-soluble organic solvent, a non-ionic surfactant, and an ester.

52. The pharmaceutical composition according to claim 51, wherein said organic polymer is PEG; said organic solvent is an amide; said alcohol is selected from the group consisting of ethanol, propanol and propylene glycol; said cyclodextrin is selected from the group consisting of hydroxypropyl-cyclodextrin and sulfobutylether-cyclodextrin; said phospholipid is selected from the group consisting of hydrogenated soy phosphatidylcholine, distearoylphosphatidylglycerol, 1-dimyristoylphosphatidylcholine, and 1-dimyristoylphosphatidylglycerol; said water-soluble organic solvent is selected from the group consisting of PEG300, ethanol, propylene glycol, glycerin, N-methyl-2-pyrrolidone, dimethylacetamide, and dimethylsulfoxide; said non-ionic surfactant is selected from the group consisting of Cremophor EL, Cremophor RH 40, Cremophor RH 60, d-tocopherol polyethylene glycol 1000 succinate, polysorbate 20, polysorbate 80, sorbitan monooleate, poloxamer 407, Labrafil M-1944CS, Labrafil M-2125CS, Labrasol, Gellucire 44/14, Softigen 767, and mono- and di-fatty esters of PEG300, PEG400 or PEG1750; and said ester is polyglycol ester.

53. The pharmaceutical composition according to claim 52, wherein said PEG is selected from the group consisting of PEG300, PEG400 and PEG1750.

54. The pharmaceutical composition according to claim 52, wherein said amide is dimethylacetamide.

55. The pharmaceutical composition according to claim 53, wherein said somatostatin-dopamine conjugate is dissolved in 20% PEG400 water solution at the concentration of about 30% (w/v); or said somatostatin-dopamine conjugate is dissolved in 5% dimethylacetamide water solution at the concentration of about 200 mg/mL; or said somatostatin-dopamine conjugate is dissolved in 5% PEG400 water solution at the concentration of about 200 mg/mL.

56. The pharmaceutical composition according to claim 1, wherein said somatostatin-dopamine conjugate is dissolved in water at the concentration range of about 15-30% (w/v).

57. The pharmaceutical composition according to claim 1, wherein the somatostatin-dopamine conjugate is present in an aqueous solution with pH between 1.0 and 10.5, preferably between 3 and 8, and more preferably between 5 and 6.

58. The pharmaceutical composition according to claim 1, wherein the somatostatin-dopamine conjugate is present in a concentration of about from 0.0001 to 500 mg/mL, preferably about from 0.1 to 300 mg/mL.

59. The pharmaceutical composition according to claim 1, further comprising a preservative, an isotonic agent, a stabilizer, a surfactant, a chelating agent, a buffer, and/or a divalent metal.

60. The pharmaceutical composition according to claim 59, wherein said preservative is selected from the group consisting of m-cresol, phenol, benzyl alcohol and methyl paraben, and is present in a concentration from 0.01 mg/mL to 100 mg/mL; said isotonic agent is present in a concentration from 0.01 mg/mL to 100 mg/mL; said stabilizer is selected from the group consisting of imidazole, arginine and histidine; said buffer is selected from the group consisting of Tris, ammonium acetate, sodium acetate, glycine, aspartic acid, and Bis-Tris; and said divalent metal is zinc.

61. A method of treating a disease or condition in a subject, said method comprising administering to said subject a therapeutically effective amount of a pharmaceutical composition according to claim 1, wherein said disease or condition is selected from the group consisting of lung cancer, glioma, anorexia, hypothyroidism, hyperaldosteronism, H. pylori proliferation, acromegaly, restenosis, Crohn's disease, systemic sclerosis, external and internal pancreatic pseudocysts and ascites, VIPoma, nesidoblastosis, hyperinsulinism, gastrinoma, Zollinger-Ellison Syndrome, diarrhea, AIDS related diarrhea, chemotherapy related diarrhea, scleroderma, Irritable Bowel Syndrome, pancreatitis, small bowel obstruction, gastroesophageal reflux, duodenogastric reflux, Cushing's Syndrome, gonadotropinoma, hyperparathyroidism, Graves' Disease, diabetic neuropathy, Paget's disease, polycystic ovary disease, thyroid cancer, hepatome, leukemia, meningioma, cancer cachexia, orthostatic hypotension, postprandial hypotension, panic attacks, GH secreting adenomas, acromegaly, TSH secreting adenomas, prolactin secreting adenomas, insulinoma, glucagonoma, diabetes mellitus, hyperlipidemia, insulin insensitivity, Syndrome X, angiopathy, proliferative retinopathy, dawn phenomenon, Nephropathy, gastric acid secretion, peptic ulcers, enterocutaneous fistula, pancreaticocutaneous fistula, Dumping syndrome, watery diarrhea syndrome, pancreatitis, gastrointestinal hormone secreting tumor, angiogenesis, arthritis, allograft rejection, graft vessel bleeding, portal hypertension, gastrointestinal bleeding, obesity, and opioid overdose.


SEQUENCE LISTING (42 sequences)

SEQ ID NO: 1 - protein, 9 aa, Artificial sequence
  Somatostatin-dopamine synthetic peptide
XXCWXKXCT

SEQ ID NO: 2 - protein, 9 aa, Artificial sequence
  Somatostatin-dopamine synthetic peptide
XXCYXKVCT

SEQ ID NO: 3 - protein, 9 aa, Artificial Sequence
  Somatostatin-dopamine synthetic peptide
XXCYXKVCT

SEQ ID NO: 4 - protein, 11 aa, Artificial sequence
  Somatostatin-dopamine synthetic peptide
XXXXCYXKVCT

SEQ ID NO: 5 - protein, 11 aa, Artificial sequence
  Somatostatin-dopamine synthetic peptide
XXXXCYXKVCT

SEQ ID NO: 6 - protein, 9 aa, Artificial sequence
  Somatostatin-dopamine synthetic peptide
XXCYXKXCT

SEQ ID NO: 7 - protein, 9 aa, Artificial sequence
  Somatostatin-dopamine synthetic peptide
XXCYXKXCT

SEQ ID NO: 8 - protein, 10 aa, Artificial sequence
  Somatostatin-dopamine synthetic peptide
XXXCYXKXCT

SEQ ID NO: 9 - protein, 9 aa, Artificial sequence
  Somatostatin-dopamine synthetic peptide
XKCYXKXCT

SEQ ID NO: 10 - protein, 11 aa, Artificial sequence
  Somatostatin-dopamine synthetic peptide
XKXXCYXKXCT

SEQ ID NO: 11 - protein, 10 aa, Artificial sequence
  Somatostatin-dopamine synthetic peptide
KXXCYXKXCT

SEQ ID NO: 12 - protein, 9 aa, Artificial sequence
  Somatostatin-dopamine synthetic peptide
XXCYXKTCT

SEQ ID NO: 13 - protein, 10 aa, Artificial sequence
  Somatostatin-dopamine synthetic peptide
XXXCYXKTCT

SEQ ID NO: 14 - protein, 9 aa, Artificial sequence
  Somatostatin-dopamine synthetic peptide
XXCYXKTCT

SEQ ID NO: 15 - protein, 11 aa, Artificial sequence
  Somatostatin-dopamine synthetic peptide
XKXXCYXKTCT

SEQ ID NO: 16 - protein, 10 aa, Artificial sequence
  Somatostatin-dopamine synthetic peptide
XKXCYXKTCT

SEQ ID NO: 17 - protein, 10 aa, Artificial sequence
  Somatostatin-dopamine synthetic peptide
XKXCYXKXCT

SEQ ID NO: 18 - protein, 10 aa, Artificial sequence
  Somatostatin-dopamine synthetic peptide
XKXCYXKXCT

SEQ ID NO: 19 - protein, 9 aa, Artificial sequence
  Somatostatin-dopamine synthetic peptide
XXCYXKXCT

SEQ ID NO: 20 - protein, 9 aa, Artificial sequence
  Somatostatin-dopamine synthetic peptide
XXCYXKXCT

SEQ ID NO: 21 - protein, 10 aa, Artificial sequence
  Somatostatin-dopamine synthetic peptide
XYXRFFXKTF

SEQ ID NO: 22 - protein, 12 aa, Artificial sequence
  Somatostatin-dopamine synthetic peptide
XKXYXRFFXKTF

SEQ ID NO: 23 - protein, 10 aa, Artificial sequence
  Somatostatin-dopamine synthetic peptide
XXXCYXKXCT

SEQ ID NO: 24 - protein, 10 aa, Artificial sequence
  Somatostatin-dopamine synthetic peptide
XXXCYXKXCT

SEQ ID NO: 25 - protein, 10 aa, Artificial sequence
  Somatostatin-dopamine synthetic peptide
XXXCYXKVCT

SEQ ID NO: 26 - protein, 9 aa, Artificial sequence
  Somatostatin-dopamine synthetic peptide
XXCYXKVCT

SEQ ID NO: 27 - protein, 9 aa, Artificial sequence
  Somatostatin-dopamine synthetic peptide
XXCYXKXCT

SEQ ID NO: 28 - protein, 13 aa, Artificial sequence
  Somatostatin-dopamine synthetic peptide
XXXKXXCYXKXCT

SEQ ID NO: 29 - protein, 9 aa, Artificial sequence
  Somatostatin-dopamine synthetic peptide
XXCYXKXCT

SEQ ID NO: 30 - protein, 9 aa, Artificial sequence
  Somatostatin-dopamine synthetic peptide
XXCYXKXCT

SEQ ID NO: 31 - protein, 9 aa, Artificial sequence
  Somatostatin-dopamine synthetic peptide
XXCYXKXCT

SEQ ID NO: 32 - protein, 12 aa, Artificial sequence
  Somatostatin-dopamine synthetic peptide
XXKXXCYXKXCT

SEQ ID NO: 33 - protein, 11 aa, Artificial sequence
  Somatostatin-dopamine synthetic peptide
XKXXCYXKXCT

SEQ ID NO: 34 - protein, 12 aa, Artificial sequence
  Somatostatin-dopamine synthetic peptide
XXKXXCYXKXCT

SEQ ID NO: 35 - protein, 11 aa, Artificial sequence
  Somatostatin-dopamine synthetic peptide
XKXXCYXKXCT

SEQ ID NO: 36 - protein, 11 aa, Artificial sequence
  Somatostatin-dopamine synthetic peptide
XXXXCYXKXCT

SEQ ID NO: 37 - protein, 11 aa, Artificial sequence
  Somatostatin-dopamine synthetic peptide
XXXXCYXKVCT

SEQ ID NO: 38 - protein, 10 aa, Artificial sequence
  Somatostatin-dopamine synthetic peptide
XXXCYXKXCT

SEQ ID NO: 39 - protein, 11 aa, Artificial sequence
  Somatostatin-dopamine synthetic peptide
XXXXCYXKVCT

SEQ ID NO: 40 - protein, 10 aa, Artificial sequence
  Somatostatin-dopamine synthetic peptide
XXXCYXKXCT

SEQ ID NO: 41 - protein, 10 aa, Artificial sequence
  Somatostatin-dopamine synthetic peptide
XXXCYXKVCT

SEQ ID NO: 42 - protein, 11 aa, Artificial sequence
  Somatostatin-dopamine synthetic peptide
XXXXCYXKXCT